SEQUENCE LISTING (2 sequences)

SEQ ID NO: 1 - DNA, 297 bp, artificial
  Design and synthesis of human epidermal growth factor gene (hEGF) nucleotide sequence
ggatccaaaattatgaaaaagacagctatcgcgattgcagtggcactggctggtttcgctaccgtagcgcaggccggcaactctgattctgaatgtccgctgtctcacgatggttactgtctgcacgatggtgtttgtatgtacatcgaagctctggataaatacgcttgtaactgcgtggtgggctacatcggtgaacgttgccagtaccgcgatctgaaatggtgggaactgcgttgataactgcagcttaattaattaagcagcccgcctaatgagcgggcttttttttctaga

SEQ ID NO: 2 - protein, 53 aa, Homo sapiens
NSDSECPLSHDGYCLHDGVCMYIEALDKYACNCVVGYIGERCQYRDLKWWELR

(19) United States
(12) Patent Application Publication
(10) Pub. No.: US 2012/0183994 A1
(43) Pub. Date: Jul. 19, 2012

(54) Vector for Expression of hEGF and Uses Thereof

(75) Inventors: Wen-Hsing Chang, Chiayi City (TW); Yi-Jiun Chen, Chiayi City (TW); Sheng-Jen Yeh, Kaohsiung (TW)

(73) Assignee: LADIES BIOTECHNOLOGY CO., LTD., Kaohsiung (TW)

(21) Appl. No.: 13/350,303

(22) Filed: Jan. 13, 2012

(30) Foreign Application Priority Data
Jan. 15, 2011 (TW) 100102659

(51) Int. Cl. C12P 21/00; C12N 15/70; C12N 15/63

(52) U.S. Cl. 435/69.1; 435/320.1

(57) ABSTRACT

A vector for expression of hEGF that is made by constructing a gene sequence of SEQ NO: 1 on an expression vector for the transformation of E. coli with said gene sequence to produce hEGF. Through said vector, high purity hEGF can be produced in E. coli and directly excreted from the cell, which means hEGF can be isolated and purified from E. coli culture. In other words, said vector for expression of hEGF can effectively simplify the production and purification procedures of hEGF and enhance its production.

### TECHNICAL FIELD OF INVENTION

This invention relates to the production of protein, and more specifically, to the production of a vector for expression of hEGF that can express and produce high purity hEGF in E.coli, with biological technology.

### BACKGROUND OF THE INVENTION

Nowadays, human life span is becoming longer and longer with the progress of medical technology; as a result, the world's aging population is increasing rapidly. Compared to the past, people nowadays not only pay attention to health but also care about whether or not they look young. Therefore, the fashion of fighting the aging process has been gradually spreading throughout the world. Take Japan as an example, more than 70% of its people have the consciousness of fighting the aging process. According to a survey made by Fuji Keiza on anti-aging cosmetics and health foods, it was estimated that by the year 2008, the total market size of Japan's anti-aging products would reach 66.69 billion yen, of which the market size of anti-aging cosmetics would reach 56.00 billion yen. In Taiwan, people's concerns about fighting the aging process have also been increasing continuously. This trend will drive the market sales of relevant anti-aging products, and it is expected the global market for these products will also expand continuously.

The discovery of EGF in recent years has unraveled the mystery of the aging process of human skin. There is EGF inside the human body. However, when people get older, the EGF content in their skin also decreases gradually, which gives rise to the phenomenon of aging featuring more and more wrinkles on their skin. Various studies suggest that supplementing EGF can resist the aging of human skin and reduce the percentage of aged cells in the skin, thus rejuvenating the skin. In other words, so long as we supplement EGF to the skin, we can fundamentally improve the texture of our skin and resist the aging process of the skin in spite of our age. Under the actions of EGF, the proliferation and differentiation of our epidermal cells will speed up, and the stem cells will also keep proliferating and differentiating into new skin cells. It is evident from this that EGF has the actions of slowing down the aging of skin cells and promoting the rehabilitation and growth of human skin cells. Therefore, it not only can smooth and brighten up our skin but also can stimulate the growth of epithelial cells and endothelial cells, promote the metabolism of various cells of the skin, help cells to absorb nutrients, promote the synthesis of collagen and elastin, thus having skin-moistening, elasticity-improving, wrinkle-reducing, and anti-aging actions.

Judging by the characteristics and advantages of EGF, it is understandable that EGF not only has actions such as promoting the healing of skin and mucosa wounds, preventing and treating ulcers, reducing inflammation, and relieving pain, but also can effectively promote and regulate the growth and proliferation f epidermal cells. Therefore, EGF has unique efficacy on protecting and nourishing the skin and mucosa. Presently, besides having been applied in various medical and pharmaceutical products, EGF also has extensive application value and market potential in skincare cosmetics. For example, cosmetics products added with EGF (generally 1 ppm to 100 ppm) can promote the forming of new skin cells, increase the contents of other endogenous growth factors, stimulate cells to excrete hyaluronic acid and glucoprotein, reasonably regulate skin structure, slow down the aging process of the skin, reduce wrinkles, inhibit the growth of pimples and acnes, and whiten the skin. EGF-containing cosmetics can also speed up the rehabilitation of wounded skin and render the skin excellent elasticity and a young and bright appearance.

In the past ten years, the rapid development of biotechnology and genetic engineering has not only led to the discovery of various new active substances but also enabled cosmetics manufacturers to effectively produce these active substances. EGF is a good example.

Also, as the result of the increase of people's income, the global aging trend, and the change of consumers' consuming habit, cosmetic products, which were categorized as luxury goods in the past, have become a basic commodity of most people in their daily life. In other words, the market of cosmetics has been expanding at an astonishing speed, and this has brought about enormous economic benefits. The cosmetics market of Taiwan, however, has been dominated by European, American, and Japanese products; and almost all cosmetics manufacturers that have a considerable market share are multinational companies. With regard to Taiwan's domestic cosmetics manufactures, most of them simply purchase materials from foreign companies, and then subpackage and sell them in Taiwan. That is to say, both the materials and the formula of their products are controlled by foreign companies.

To put it more specifically, that is, Taiwan depends on foreign companies for most of its cosmetics materials, materials for medicine or medical cosmetology, and production technology. Take the development and production of EGF as an example. Presently EGF is no longer obtained only by extraction; rather, in order to achieve higher production efficiency, it is first extracted and then cloned. Presently, Taiwan's domestic manufacturers still have to purchase from foreign companies the EGF they needed which, in spite of its high quality and activity, is sold at high prices. Some manufacturers also produce EGF using clone technology, and this does effectively reduce the production cost. However, in most cases the EGF they produce is not stable enough; as a result, the EGF they produce varies in terms of activity and fails to achieve expected efficacy, thus impairing the competitiveness of their products.

### SUMMARY OF THE INVENTION

In the light of the above-mentioned facts, the main purpose of this invention is to provide a vector for expression of hEGF. A gene sequence of SEQ NO:1 is attached to a trisystem vector system and then transferred into a host to express hEGF. The host can be E. coli.

More specifically, the vector comprises:
a promotor capable of expressing a protein in a bacterium regulated by iopropyl β-D-thiogalactoside(IPTG) to induce protein expression;
a gene sequence of SEQ No: 1 linked successively after the promotor; and
a sequence consisting of 8 histdines linked successively after the gene sequence of SEQ NO: 1;
a section of OmpA-containing inducible secretion signal peptide sequence linked before the gene sequence of SEQ NO: 1 that enables the downstream protein to be excreted from the host.

Another purpose of this invention is to provide a vector for the expression of hEGF that may express in the host a large amount of hEGF; wherein the host is E. coli.

Yet another purpose of this invention is to provide a use of the hEGF-containing vector, which enables the experimenter to carry out direct bacterial lysis to obtain and purity the supernatant to get high purity hEGF, thereby effectively cutting down the cost of protein purification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is the pQE-TriSystem vector.

FIG. 2 shows the electrophoresis of the clone of the pQE-Trisystem with EGF gene construction

FIG. 3 shows the protein expression results induced by IPTG at various concentrations for 4 hours, wherein M is the protein marker.

FIG. 4 shows the expression results induced by 1 mM IPTG at 0, 1, 2, 3, and 4 hours, wherein M is the protein marker.

FIG. 5 is the SDS-PAGE analysis of the purified EGF protein, wherein M is the protein marker.

FIG. 6 is the SDS-PAGE analysis of the hEGF purified with a nickel column.

FIG. 7 is the MTT assay of the activity of hEGF at various concentrations

FIG. 8 is a statistical histogram of the activity of human epidermal cells.

### DETAILED DESCRIPTION OF THE PREFERRED EMBODIMENTS

This invention discloses a vector for expression of hEGF that is made by constructing a gene sequence of SEQ NO: 1 on an expression vector for the transformation of E. coli with said gene sequence of SEQ NO: 1 to produce hEGF. Through said vector, high purity hEGF can be produced in E. coli and directly excreted from the cell, which means hEGF can be isolated and purified from E. coli culture. In other words, the vector for expression of hEGF can effectively simplify the production and purification procedures of hEGF and enhance its production.

The efficacy of this invention will be further illustrated with the following examples given in conjunction with the accompanying drawings.

### EXAMPLE 1

### Construction of the Expression Vector

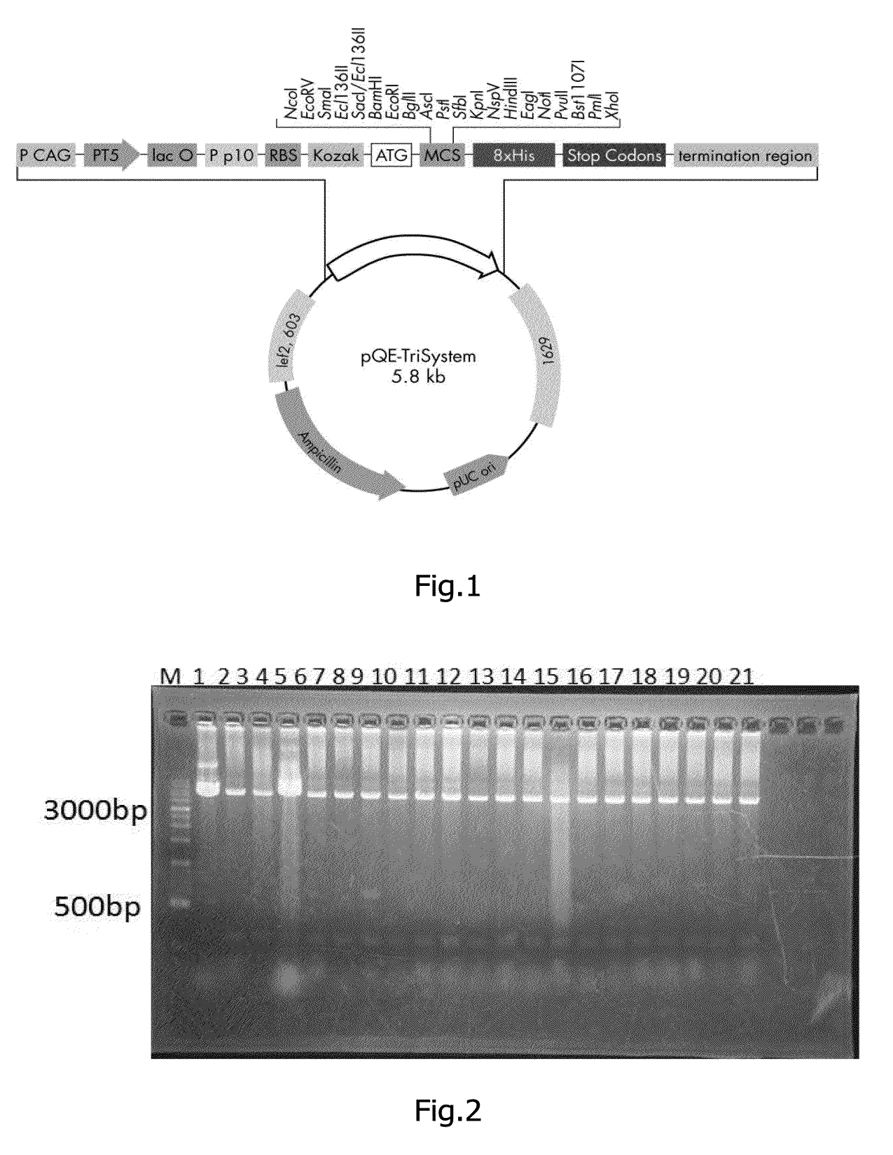

First, a gene sequence of SEQ NO: 1 containing a functional gene sequence segment of SEQ NO: 2 of human epidermal growth factor (hEGF) is produced using DNA synthesizer. The gene sequence of SEQ NO: 1 is then spliced into a pKS plasmid. Upon confirmation of the synthetic DNA sequence with genetic analyzer, the plasmid is cloned to a pQE-trisystem vector, as shown in FIG. 1, to form a pQE-trisystem-hEGF vector, wherein the pQE-trisystem-hEGF vector contains a promoter whose inducible expression can be regulated by isopropyl-β-D-thiogalactoside (IPTG) to get a expression protein inside bacteria; the gene sequence of SEQ NO: 1 is linked after the promoter; a sequence of 8 histidines is linked after the gene sequence of SEQ NO: 1 successively, serving as a tag for purification of recombinant EGF-8X His fusion protein. An OmpA-containing inducible secretion signal peptide sequence is linked before the gene sequence of SEQ NO: 1 to secrete downstream proteins out of the bacteria, enabling the experimenter to obtain the supernatant for purification by means of direct bacterial lysis to get recombinant EGF-8X His fusion protein.

### EXAMPLE 2

### Implementation of Gene Transformation

The pQE-trisystem-hEGF vector constructed in example 1 is electroporated or treated with CaCl2, and transferred into E. coli. Cut the pQE-trisystem-hEGF vector with kanI and NcoI restriction enzymes, and then use SDS PAGE to analyze whether hEGF is expressed, and the results are as show in FIG. 2, wherein M is the marker, 1˜21 represent different clones. It can be observed in FIG. 2 that the expected fragment sizes are 5800 by and 200 bp.

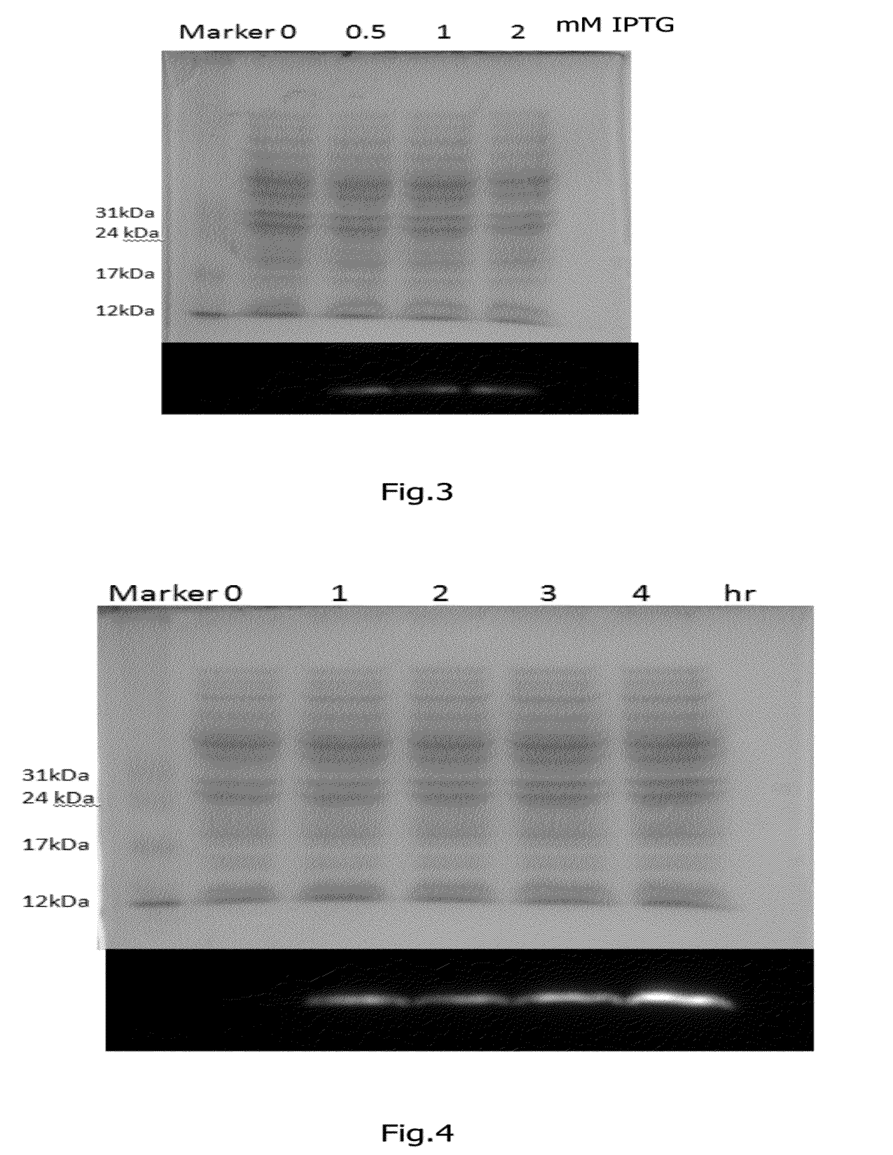

Culture the transgenic E. coli at specific conditions for a scheduled time, then induce the E. coli with IPTG to enable the expression vector to express hEGF in E. coli. Determine the application concentration and induction time of IPTG with SDS-PAGE to achieve the highest production efficiency, and the results are as shown in FIG. 3 and FIG. 4. FIG. 3 shows the results of hEGF expression induced by IPTG for 4 hours at various concentrations; FIG. 4 shows the results of hEGF expression induced by 1 mM IPTG for 0, 1, 2, 3, and 4 hours respectively. It can be inferred from the above Figures that the optimum production efficiency is achieved by the induction of expression with 1 mM IPTG for 4 hours.

### EXAMPLE 3

Purification of hEGF

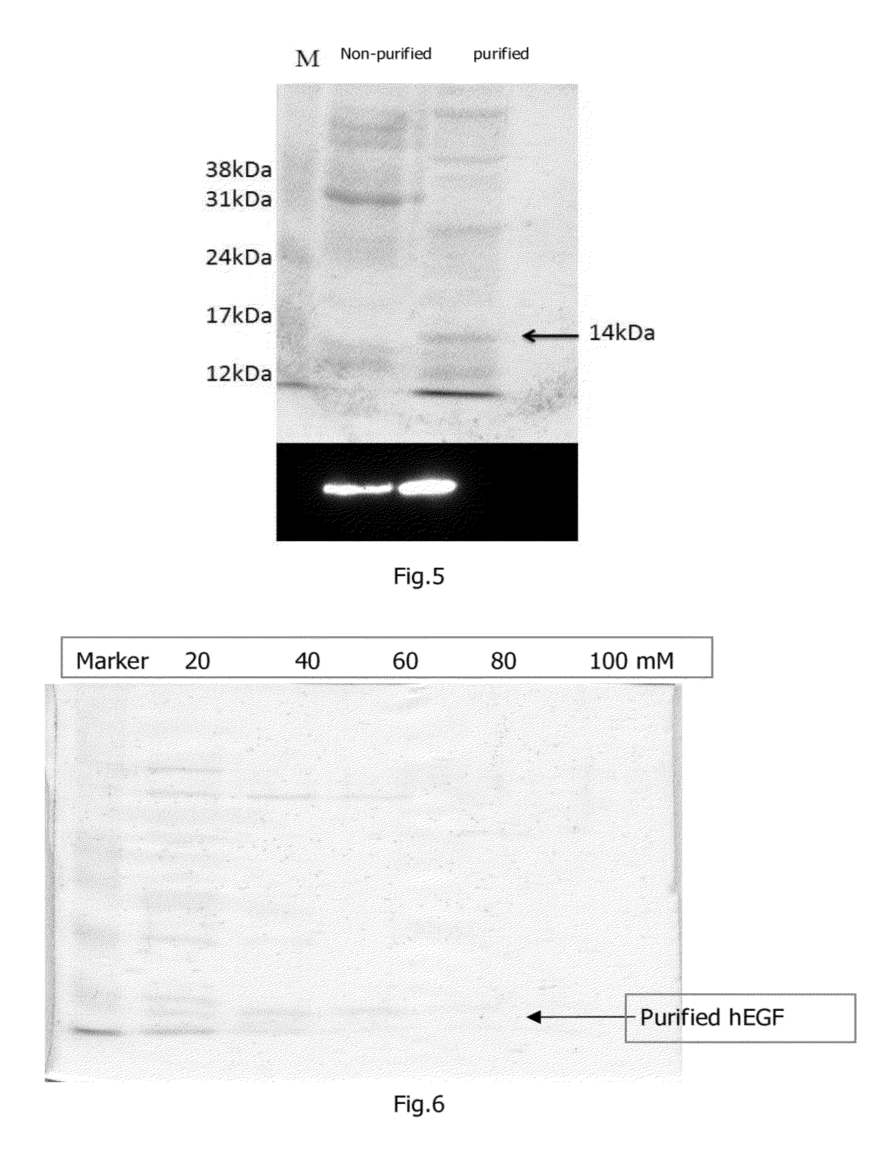

The expression vector of Example 1 can be used to excrete recombinant EGF-8X His fusion protein from the cell; therefore, in Example 2, the E. coli successfully transformed will secrete the fusion protein out of the cells, which enables the experimenter to carry out direct bacterial lysis and centrifugation successively to remove the supernatant, which is then underwent fractional extraction with imidazole. The recombinant EGF-8X His fusion protein obtained is analyzed with SDS-PAGE, with results as shown in FIG. 5. It can be inferred from the figure that the purity of the extract can increase 5-10 times by treating the supernatant with imidazole.

This is because the recombinant EGF-8X His fusion protein expressed by the expression vector obtained in Example 1 contains 8X His tag and can bind with nickel. In order to get EGF of purity higher than 90%, it is necessary to purify the recombinant protein using an affinity chromatography column. Therefore, the recombinant EGF-8X His fusion protein is purified using a nickel column and then analyzed with SDS-PAGE, and the results are as shown in FIG. 6. It can be inferred from the figure that after the hEGF in the crude extract is purified using a nickel column, only a single band is left in the electrophoresis when the salinity of the electrophoretic liquid is 80 nM. That is to say, the purity of the recombinant hEGF is higher than 90%.

### EXAMPLE 4

Activity of the hEGF Purified with Chromatography

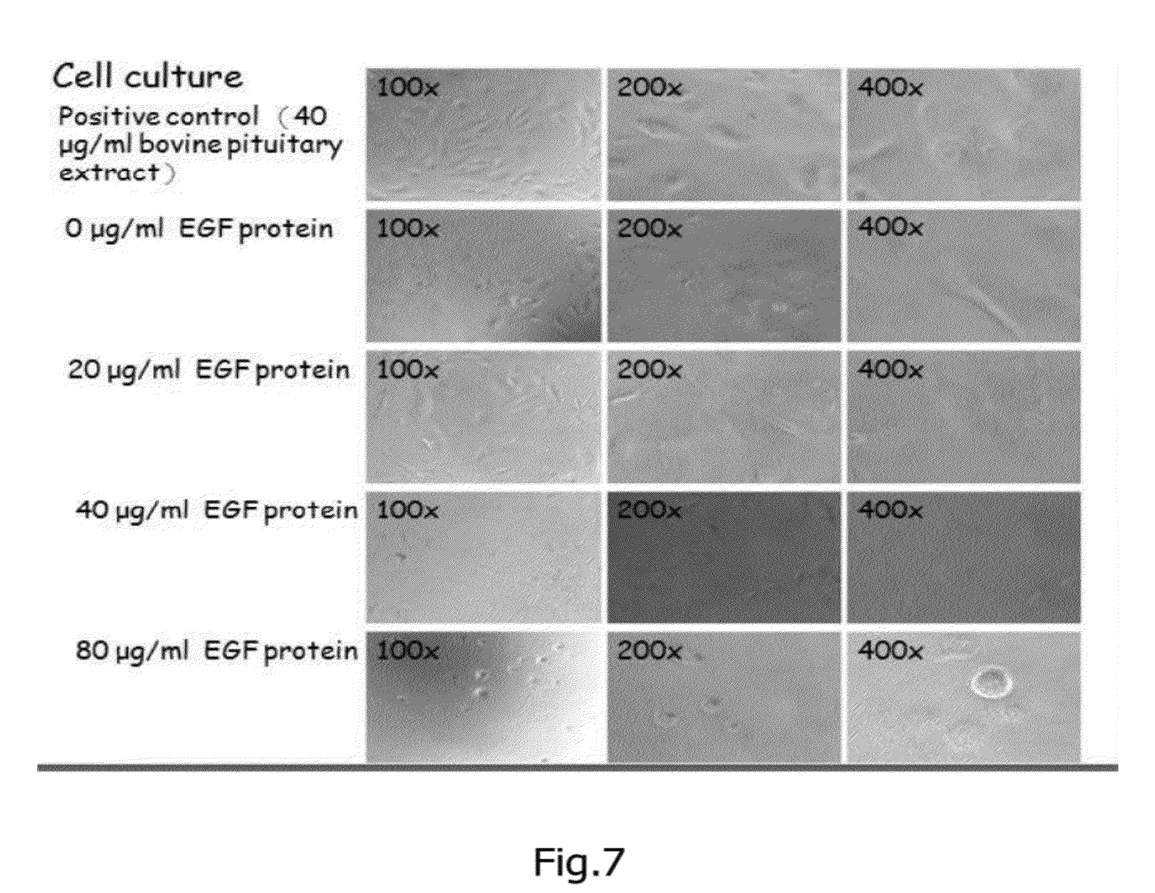

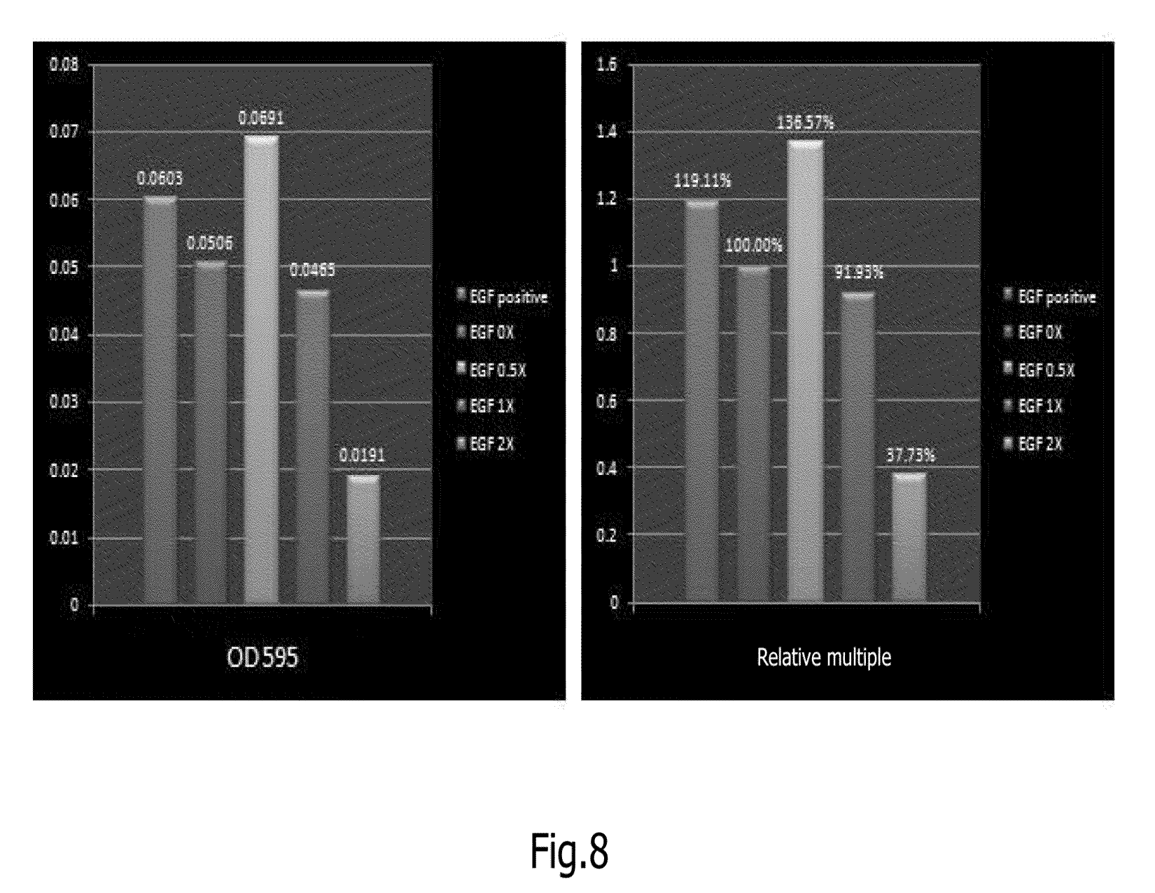

First, human epidermal cell lines are cultured in a culture solution, into which the purified hEGF of Example 3 is added at various concentrations (0 ug/ml·20 ug/ml·40 ug/ml·80 ug/ml). After 2-7 days, the increased amount of epidermal cells are determined by MTT assay, then statistics is carried out on the activity of the epidermal cells, so as to find out whether or not the purified hEGF can promote the proliferation of the epidermal cells, and the results are as shown in FIG. 7 and FIG. 8. It can be inferred from the figures that the said hEGF can promote the proliferation of human epidermal cells.

It can also be inferred from the above-disclosed Examples 1 through 4 that the vector for expression of hEGF of this invention does express in the E. coli a large amount of recombinant hEGF which can be isolated from the culture solution and purified directly because the said recombinant hEGF is excreted from the cells. Therefore, it can substantially reduce the purification process and procedures, effectively cut down production cost, and enhance production.

The foregoing description and Examples detailing certain preferred embodiments of the invention are to be considered as illustrative and not restrictive, and the invention is not to be limited to the details given herein, but may be modified within the scope and equivalents of the appended claims.

What is claimed is:

1. A vector for expression of hEGF that is made by constructing a gene sequence of SEQ NO: 1 on an expression vector used for the transformation of a host to express hEGF.

2. The vector for expression of hEGF of claim 1, wherein the host is E. coli.

3. A vector for expression of hEGF comprising:
a promotor expressing a protein in a bacterium regulated by iopropyl β-D-thiogalactoside(IPTG) to induce protein expression;
a gene sequence of SEQ No: 1 linked after the promotor; and
a sequence consisting of 8 histdines linked successively after the gene sequence of SEQ NO: 1.

4. The vector for expression of hEGF of claim 3, wherein a section of OmpA-containing inducible secretion signal peptide sequence is linked before the gene sequence of SEQ NO: 1 used for secreting downstream protein out of the bacterium.

5. A use of the vector of claim 3 is to express in a host a large amount of high purity hEGF.

6. The use of the vector of claim 5, wherein the host is E. coli.
